(19) United States
(12) Patent Application Publication
(10) Pub. No.: US 2015/0080387 A1
(43) Pub. Date: Mar. 19, 2015

(54) MEDICAMENT FORMULATIONS COMPRISING FLUOROQUINOLONES

(71) Applicant: BAYER INTELLECTUAL PROPERTY GMBH, Monheim (DE)

(72) Inventors: Iris HEEP, Koln (DE); Kristine FRAATZ, Burscheid (DE); Hans-Jürgen HAMANN, Dormagen (DE); Markus EDINGLOH, Leverkusen (DE)

(21) Appl. No.: 14/550,877

(22) Filed: Nov. 21, 2014

Related U.S. Application Data
(63) Continuation of application No. 12/280,996, filed on Oct. 31, 2008.

(30) Foreign Application Priority Data
Mar. 8, 2006 (DE) 102006010642.3

(51) Int. Cl. A61K 47/02; A61K 31/496; A61K 45/06; A61K 31/4709

(52) U.S. Cl. CPC A61K 47/02; A61K 31/4709; A61K 31/496; A61K 45/06
USPC 514/229.2; 514/300; 514/253.08

(57) ABSTRACT

The invention relates to pharmaceutical formulations in liquid form, containing fluoroquinolones and antioxidative sulphur compounds. The formulations are particularly suitable for parenteral uses and are distinguished, inter alia, by good tolerance.

The invention relates to pharmaceutical formulations in liquid form comprising fluoroquinolones and antioxidant sulphur compounds. The formulations are particularly suitable for parenteral administrations and are distinguished inter alia by the fact that they are well tolerated.

The chemical stability of solutions can be increased for example by using antioxidants. The oxidative degradation of a constituent can thereby be prevented. This is also customary in solutions for injection, where this is particularly the case. Antioxidants which are conventionally used here are, inter alia, the sulphites. DE-A-19500784, EP-A-0187315 or EP-A-1121933 describe solutions for injection which contain sulphites. Eye drops too, when present as solutions, are provided with sulphites, as this is described in EP-A-0804267. DE-A-2364470 describes the use of sulphites which is intended to prevent decoloration of the formulation. The use of such sulphites for improving the local tolerance of solutions for injection has not been described as yet. Solutions which are not well tolerated must, in practice, be administered intravenously, for example in the form of an infusion. However, the practice of this procedure is problematic, in particular when animals are treated. There has also been a number of attempts to increase the tolerance for example by formulating the product as liposomes, as this is described in WO 98/33482. Cyclodextrins also are a possibility, frequently studied, in order to improve the solubilities or tolerances of formulations; see EP-A-0209768. If the tolerance of a formulation cannot be improved at all, it may be necessary to use a local anaesthetic when applying it, as this is described in GB-A-1143330; or to use oily formulations instead; EP-A-1121933.

Solutions for parenteral administration on animals are special in as far as they must be applied in different ways and means, depending on the animal species. For example, it is conventional practice in Europe to administer solutions for injection subcutaneously to pigs and intramuscularly to dogs or cats. Increased tolerance requirements must be met not only as the result of the animal species, but also as the result of the different routes of administration (EP-A-1121933). The fact that they are tolerated by cattle, for example, does not necessarily allow the conclusion that they are tolerated by, for example, cats or dogs (WO 01/81358). In order to ensure a broad applicability, it is therefore meaningful to improve the local tolerance of solutions for injection in such a way that they can be used even in sensitive animal species.

It is therefore also not surprising that most solutions for injection which contain fluoroquinolones are not available for dogs or cats, the reason being, inter alia, that they are not well tolerated.

To ensure that the solutions are as well tolerated as possible, it is recommended to maintain their pH as neutral as possible (approx. 7.4), which, however, is in contrast with the fluoroquinolones' solubility. Also, particle formation of the betaine form of the fluoroquinolones can frequently be observed in this pH range, which is why solutions, while tolerated, have a short shelf life and particle formation results. This can be avoided for example by choosing freeze-dried products instead. Freeze-dried products, however, are difficult to handle in practice and frequently only have a shelf life, of the reconstituted solution, of no more than 4 weeks after reconstitution, or must be discarded directly as the result of the possibility of particle formation. Accordingly, a ready-to-use solution is advantageous as solution for injection.

It is furthermore necessary that a suitable amount of the fluoroquinolone enters the serum after the administration, as this is also described in WO 99/29322. Again, this is not a matter of course with injectable fluoroquinolone formulations and may likewise depend on the animal species in question.

There have been found ready-to-use injectable formulations containing fluoroquinolones which comprise sufficient concentration of the fluoroquinolone, which are stable and free from particle formation upon storage under pharmaceutical conditions, which are well tolerated, in particular by dogs, and which have advantageous serum kinetics.

The invention therefore relates to:

a pharmaceutical formulation in liquid form containing:
(a) a fluoroquinolone,
(b) an antioxidative sulphur compound
(c) if appropriate, further pharmaceutical auxiliaries and/or additives

Fluoroquinolones are, inter alia, compounds as they are disclosed in the following documents: U.S. Pat. No. 4,670,444 (Bayer A G), U.S. Pat. No. 4,472,405 (Riker Labs), U.S. Pat. No. 4,730,000 (Abbott), U.S. Pat. No. 4,861,779 (Pfizer), U.S. Pat. No. 4,382,892 (Daiichi), U.S. Pat. No. 4,704,459 (Toyama), the following being mentioned as specific examples: benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, pefloxacin, pipemidic acid, temafloxacin, tosufloxacin, sarafloxacin, sparfloxacin.

A preferred group of fluoroquinolones are those of the formula (I) or (II):

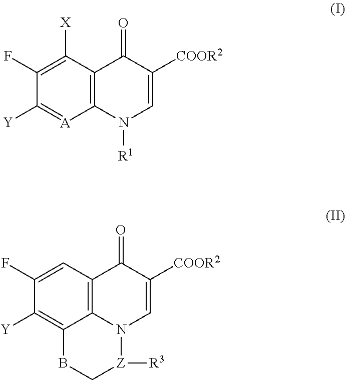

in which
X represents hydrogen, halogen, C₁₋₄-alkyl, C₁₋₄-alkoxy, NH₂,
Y represents radicals of the structures

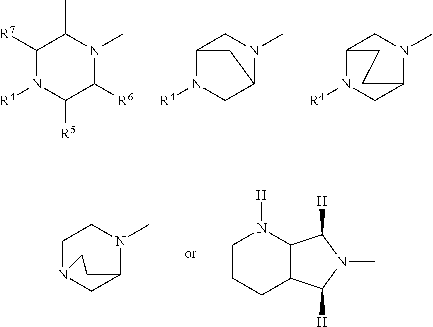

in which
R⁴ represents optionally hydroxyl- or methoxy-substituted straight-chain or branched C₁-C₄-alkyl, cyclopropyl, acyl having 1 to 3 C atoms,
R⁵ represents hydrogen, methyl, phenyl, thienyl or pyridyl,
R⁶ represents hydrogen or C₁₋₄-alkyl,
R⁷ represents hydrogen or C₁₋₄-alkyl,
R⁸ represents hydrogen or C₁₋₄-alkyl,
   and
R¹ represents an alkyl radical having 1 to 3 carbon atoms, cyclopropyl, 2-fluoroethyl, methoxy, 4-fluorophenyl, 2,4-difluorophenyl or methylamino,
R² represents hydrogen or optionally methoxy- or 2-methoxyethoxy-substituted alkyl having 1 to 6 carbon atoms and cyclohexyl, benzyl, 2-oxopropyl, phenacyl, ethoxycarbonylmethyl, pivaloyloxymethyl,
R³ represents hydrogen, methyl or ethyl and
A represents nitrogen, ═CH—, ═C(halogen)-, ═C(OCH₃)—, ═C(CH₃)— or ═C(CN),
B represents oxygen, optionally methyl- or phenyl-substituted ═NH or ═CH₂,
Z represents ═CH— or ═N—,
   and their pharmaceutically useful salts and hydrates.
   Preferred compounds of the formula (I) are those
   in which
A represents ═CH— or ═C—CN,
R¹ represents optionally halogen-substituted C₁-C₃-alkyl or cyclopropyl,
R² represents hydrogen or C₁₋₄-alkyl,
Y represents radicals of the structures

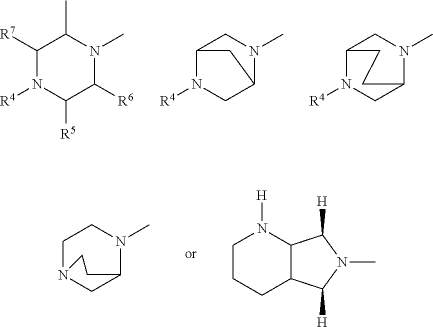

in which
R⁴ represents optionally hydroxyl-substituted straight-chain or branched C₁-C₃-alkyl, oxalkyl having 1 to 4 C atoms,
R⁵ represents hydrogen, methyl or phenyl,
R⁶ represents hydrogen,
R⁷ represents hydrogen or methyl,
R⁸ represents hydrogen,
   and their pharmaceutically useful hydrates and salts.
   Especially preferred compounds of the formula (I) are those
   in which
A represents ═CH— or ═C—CN,
R¹ represents cyclopropyl,
R² represents hydrogen, methyl or ethyl,
Y represents radicals of the structures

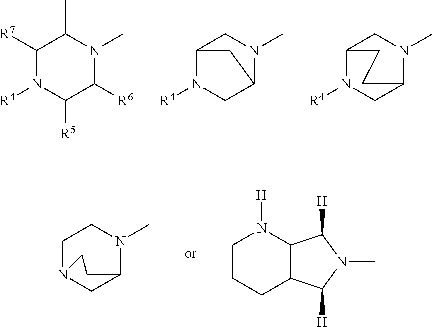

in which
R⁴ represents methyl, optionally hydroxyl-substituted ethyl,
R⁵ represents hydrogen or methyl,
R⁶ represents hydrogen,
R⁷ represents hydrogen or methyl,
R⁸ represents hydrogen,
   and their pharmaceutically useful salts and hydrates.
   A preferred example of a fluoroquinolone of the formula (II) which may be mentioned is marbofloxacin:

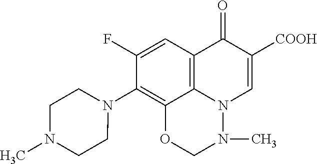 [SMILES: CN1CCN(c2c(F)cc3c(=O)c(C(=O)O)cn4c3c2OCN4C)CC1]

Especially preferred fluoroquinolones which may be mentioned are the compounds described in WO 97/31001, in particular 8-cyano-1-cyclopropyl-7-((1S,6S)-2,8-diazabicyclo[4.3.0]nonan-8-yl)-6-fluoro-1,4-dihydro-4-oxo-3-quinolinecarboxylic acid (pradofloxacin), of the formula

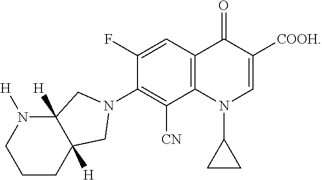 [SMILES: N#Cc1c(N2C[C@@H]3CCCN[C@@H]3C2)c(F)cc2c(=O)c(C(=O)O)cn(C3CC3)c12]

Enrofloxacin is also especially preferably employed: 1-cyclopropyl-7-(4-ethyl-1-piperazinyl)-6-fluoro-1,4-dihydro-4-oxo-3-quinolinecarboxylic acid

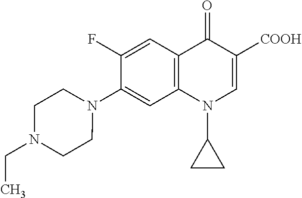 [SMILES: CCN1CCN(c2cc3c(cc2F)c(=O)c(C(=O)O)cn3C2CC2)CC1]

The use of ciprofloxacin, an active substance usually employed in human medicine, is also feasible.

Optically active fluoroquinolones can exist in the form of their racemates or in enantiomeric forms. Not only the pure enantiomers, but also their mixtures can be employed in accordance with the invention.

Suitable salts are pharmaceutically useful acid addition salts and basic salts.

Pharmaceutically useful salts are taken to mean, for example, the salts of hydrochloric acid, sulphuric acid, acetic acid, glycolic acid, lactic acid, succinic acid, citric acid, tartaric acid, methanesulphonic acid, 4-toluenesulphonic acid, galacturonic acid, gluconic acid, embonic acid, glutamic acid or aspartic acid. Furthermore, the compounds according to the invention can be bound to acidic or basic ion exchangers. Pharmaceutically useful basic salts which may be mentioned are the alkali metal salts, for example the sodium or potassium salts, the alkaline earth metal salts, for example the magnesium or calcium salts; the zinc salts, the silver salts and the guanidinium salts.

Hydrates are taken to mean not only the hydrates of the fluoroquinolones themselves, but also the hydrates of their salts. An example which may be mentioned is pradofloxacin, which forms a stable trihydrate (see WO 2005/097789).

Fluoroquinolones, being solids, can, under certain circumstances, form various crystal modifications. Advantageous for the pharmaceuticals of the present invention are those modifications which have suitable solubility properties.

The fluoroquinolone is typically employed in an amount, for animals with a body weight of up to approximately 80 kg, of 0.1 to 15%, preferably 0.5 to 15% and especially preferably 1 to 15%. In the case of animals with a body weight of more than approximately 80 kg, the fluoroquinolone is typically employed in an amount of from 1 to 30%, preferably 3 to 25% and especially preferably 4 to 20%. The data in percentages are given, in each case, as w/v.

Examples of antioxidant sulphur compounds are: sulphites (sodium sulphite, potassium sulphite), bisulphites (such as, for example sodium metabisulphite, potassium metabisulphite, potassium pyrosulphite, sodium pyrosulphite, acetosodium metabisulphite, acetosodium bisulphite), thiosulphates (such as, for example, potassium thiosulphate, sodium thiosulphate), and organic sulphur compounds (such as, for example, sodium formaldehyde sulphoxylate, thiourea, thiosorbitol, cysteine hydrochloride, cystine, cysteine, acetylcysteine, glutathione, cysteamine, methionine, thioglycerol, thioglycolic acid, thiolactic acid).

The antioxidant sulphur compounds are usually employed in concentrations of from 0.05 to 10%, preferably of from 0.1 to 8% and especially preferably of from 0.5 to 5%. The data in percentages are in each case given as w/v.

The liquid formulations can contain further substances which improve the local tolerance upon application. Examples which may be mentioned are: free-radical scavengers or antioxidants such as, for example, vitamin E, water-soluble vitamin E esters or vitamin C, butylhydroxyanisole or butylhydroxytoluene. Complexing agents such as, for example sodium-EDTA (ethylenediaminetetraacetic acid), polyvinylpyrrolidone or cyclodextrins, in particular in the text below, hydroxypropyl-β-cyclodextrin or sulphobutylether-β-cyclodextrin, dexpanthenol, salts of fatty acids such as, for example, sodium caprylate, salts of polyvalent cations (for example of the alkaline earth metals Me²⁺ or Me³⁺) and here in particular magnesium in its salt forms, amino acids and here particularly arginine or lysine, poloxamers, poloxamines, cosolvents such as, for example, n-butanol, glycerol, polyethylene glycol, propylene glycol or dimethylacetamide, dextrans, acids such as, for example, gluconolactonic acid, lactic acid, embonic acid, citric acid, tartaric acid, mucic acid or hyaluronic acid, lecithins with a phosphatidylcholine content of 70-100% from soya or chicken protein or else creatinine

Substances which improve the tolerance are usually present in concentrations of from 0.05 to 10%, preferably from 0.1 to 8% and especially preferably 0.5 to 5%. The data in percentages are given, in each case, as w/v.

Substances which are capable of preventing particle formation are, for example, poloxamers, lecithins, polyvinylpyrrolidones, cosolvents, antioxidants, complexing agents or else quaternary ammonium compounds such as, for example, benzethonium chloride or benzalkonium chloride.

Substances which are capable of improving the stability and of avoiding for example particle formation are usually employed in concentrations of from 0.001 to 10%, preferably at 0.005 to 6% and especially preferably at 0.001 to 3%. The data in percentages are given, in each case, as w/v.

The solvent which the liquid formulation may contain is water or water-miscible substances. An example which may be mentioned are glycerol, propylene glycol, polyethylene glycols, tolerated alcohols such as ethanol, benzyl alcohol or n-butanol, ethyl lactate, ethyl acetate, triacetin, N-methylpyrrolidone, propylene carbonate, propylene glycol, glycofurol, dimethylacetamide, 2-pyrrolidone, isopropylidene glycerol, or glycerin formal. Mixtures of various solvents may also be used. Water-based formulations, which naturally may also contain further solvents and cosolvents, are preferred.

Besides water or water-miscible substances, the liquid formulation may also contain oils in the form of an emulsion as solvent. Among these, substances which may be mentioned are the vegetable, animal and synthetic oils such as cottonseed oil, sesame oil, soya oil, medium-chain triglycerides with a chain length of C₁₂-C₁₈, propylene glycol octanoate/decanoate or else paraffin.

The solvent is usually employed at concentrations of from 99.8 to 72% or 98.9 to 55%, respectively, preferably at 99.4 to 81% or 96.9 to 67%, respectively, and especially preferably at 98.8 to 87% or 94.5 to 77%, respectively. The data in percentages are given, in each case, as w/v.

The pH of the liquid formulations is usually 2-11, preferably 3-8 and especially preferably 4-8.

The pharmaceuticals may also contain cosolvents, and here preferably in those cases when the formulations contain water. These are usually employed in amounts of from 1 to 10% by weight, preferably 3 to 8%. Examples of cosolvents which may be mentioned are: pharmaceutically tolerated alcohols, dimethyl sulphoxide, ethyl lactate, ethyl acetate, triacetin, N-methylpyrrolidone, propylene carbonate, propylene glycol, glycofurol, dimethylacetamide, 2-pyrrolidone, isopropylidene glycerol, glycerine formal, glycerin and polyethylene glycols. Substances which are suitable as cosolvent are, in particular, pharmaceutically acceptable alcohols such as, for example, ethanol, benzyl alcohol or n-butanol. Mixtures of the abovementioned solvents may also be employed as cosolvent.

The liquid formulation may contain preservatives, for example aliphatic alcohols such as benzyl alcohol, ethanol, n-butanol, phenol, cresols, chlorobutanol, para-hydroxybenzoic esters (in particular the methyl and propyl esters), salts or the free acids of the carboxylic acids, such as sorbic acid, benzoic acid, lactic acid or propionic acid, benzalkonium chloride, benzethonium chloride or cetylpyridinium chloride.

Depending on the type of formulation and on the form of administration, the pharmaceuticals according to the invention may contain further customary, pharmaceutically acceptable additives and adjuvants. Examples which may be mentioned are:
antioxidants such as, for example, phenols (tocopherols, and also vitamin E and vitamin-E-TPGS (d-alpha-tocopheryl polyethylene glycol 1000 succinate)), butylhydroxyanisole, butylhydroxytoluene, octyl and dodecyl gallate), organic acids (ascorbic acid, citric acid, tartaric acid, lactic acid) and their salts and esters,
wetting agents such as, for example, salts of fatty acids, or fatty alkyl sulphates, fatty alkyl sulphonates, linear alkylbenzene sulphonates, fatty alkyl polyethylene glycol ether sulphates, fatty alkyl polyethylene glycol ethers, alkylphenol polyethylene glycol ethers, alkyl polyglycosides, fatty acid N-methylglucamides, polysorbates, sorbitan fatty acid esters and poloxamers.
Iso-osmotics, such as, for example, sodium chloride, glucose or glycerol.
Pharmaceutically acceptable colorants such as, for example, iron oxides, carotenoids and the like.

In addition to the fluoroquinolones, the formulations according to the invention may comprise further pharmaceutical active ingredients. For example, the fluoroquinolones may also be employed in combination with, for example, pain killers, in particular what are known as NSAIDs (nonsteroidal antiinflammatory substances). Such NSAIDs may be, for example: meloxicam, flunixin, ketoprofen, carprofen, metamizole or (acetyl)salicylic acid.

The pharmaceuticals according to the invention can be prepared by dispersing the fluoroquinolone after the antioxidative sulphur compound in the solvent and other substances for improving tolerance and, if appropriate, for avoiding particle formation are likewise added. Cosolvents and further constituents such as, for example, preservatives can already be added to the solvent or else admixed later.

Alternatively, cosolvents, preservatives, substances which influence the tolerance or the formation of particles may also first be dissolved in the solvent and the mixture is only then complemented by the fluoroquinolone. The antioxidative sulphur compound may also be dispersed with or after the fluoroquinolone.

In general, the pharmaceutical preparations according to the invention are suitable for use in humans and animals. They are preferably employed in animal keeping and animal husbandry in livestock, breeding animals, zoo animals, laboratory animals, experimental animals and pets.

The livestock and breeding animals include mammals such as, for example, cattle, horses, sheep, pigs, goats, camels, water buffalos, donkeys, rabbits, fallow deer, reindeer, fur bearers such as, for example, minks, chinchilla, racoons and birds such as, for example, chickens, geese, turkeys, ducks, pigeons and bird species for keeping on domestic premises and in zoos.

The laboratory and experimental animals include mice, rats, guinea pigs, golden hamsters, dogs and cats.

The pets include rabbits, hamsters, guinea pigs, mice, horses, reptiles, suitable bird species, dogs and cats.

Fish may also be mentioned, and here useful fish, farmed fish, aquarium fish and ornamental fish of all ages which live in fresh water and sea water.

The preparations according to the invention are preferably employed in pets such as horses, cats and dogs. They are particularly suitable for use in cats and dogs.

Examples of preferred livestock are cattle, sheep, pig, goat and chicken. Especially preferred livestock is cattle and pig.

The administration can be effected prophylactically or else therapeutically.

The formulations described herein can be administered to the target organism (human or animal) via different routes, for example, they can be administered parenterally, in particular by means of an injection (for example subcutaneously, intramuscularly, intravenously, intramammarially, intraperitoneally), dermally, orally, rectally, vaginally or nasally, with parenteral administration—in particular by means of an injection—being preferred.

The formulations are preferably administered as solutions, suspensions or emulsions.

The pharmaceuticals according to the invention are distinguished by good stability and good solubility of the active substance. Moreover, they have good tolerance and suitable serum kinetics in animals, in particular upon parenteral administration.

### EXAMPLES

The formulations of the following examples are prepared by mixing or dissolving the stated ingredients in water for injection. The pH of the solutions can be adjusted by addition of acids or bases. The solutions for injection are filter-sterilized and transferred into suitable containers. Pradofloxacin can be employed as the anhydrate or as the trihydrate; the numerical values are calculated in each case for the anhydrate.

(Percentages in percent by weight based on the total volume of the finished preparation, [w/v]).

### Example 1

3.0% pradofloxacin
0.1% poloxamer F68
0.2% sodium disulphite
3% n-butanol
1.6% sodium hydroxide (1N)
2.7% sodium chloride
water for injection to 100%

3.0 g of pradofloxacin, 0.1 g of poloxamer, 0.2 g of sodium disulphite, 3 g of n-butanol, 2.7 g of sodium chloride are dissolved in approximately 80 g of water for injection, the pH is checked and, if necessary, brought to pH 7.4 with 1.6 g of 1N sodium hydroxide. Thereafter, the remainder of the water for injection is added to give the final volume of 100 ml.

### Example 2

3% pradofloxacin
0.1% poloxamer F68
0.5% sodium disulphite
3% n-butanol
2.4% sodium chloride
4.6% 1N sodium hydroxide
water for injection to 100%

3 g of pradofloxacin, 0.1 g of poloxamer, 0.5 g of sodium disulphite, 3 g of n-butanol, 2.4 g of sodium chloride are dissolved in approximately 80 g of water for injection, the pH is checked and brought to pH 7.4 with 4.6 g of sodium hydroxide. Thereafter, the remainder of the water for injection is added to give the final volume of 100 ml.

### Example 3

3% pradofloxacin
0.2% sodium disulphite
3% n-butanol

### 20% N-methylpyrrolidone

water for injection to 100%

70 g of water for injection are mixed with 3 g of n-butanol and 20 g of N-methylpyrrolidone. 0.2% of sodium disulphite are dissolved in this mixture, followed by 3 g of pradofloxacin. The remaining 3.8 g of the water for injection is added to give the final volume of 100 ml.

### Example 4

1% enrofloxacin
0.5% sodium disulphite
1.4% benzyl alcohol

### 1.4 g 1N KOH

3% magnesium chloride hexahydrate
water for injection to 100%

70 g of water for injection are mixed with 1.4 g of benzyl alcohol and 3 g of magnesium chloride hexahydrate. 0.5% sodium disulphite with 1.4 g of 1N KOH are dissolved in this mixture, followed by 1 g of enrofloxacin. The remaining 22.7 g of the water for injection is added to give the final volume.

### Example 5

5% pradofloxacin (trihydrate)
0.1% poloxamer F68
3% n-butanol
0.5% sodium disulphite
9% 1N hydrochloric acid
water for injection to 100%

80 g of water for injection are mixed with 0.5 g of sodium disulphite, 3 g of n-butanol and 0.1 g of poloxamer. 5 g of pradofloxacin (trihydrate, calculated as pure pradofloxacin) are dissolved in this mixture. If necessary, a pH of 5 is set with approximately 9 g of acid, and the mixture is brought to the final volume of 100 ml with the remaining water for injection.

### Example 6

2% pradofloxacin (trihydrate)
0.1% poloxamer F68
3% n-butanol
0.5% sodium disulphite
2.6% sodium chloride
9% 1N sodium hydroxide
water for injection to 100%

80 g of water for injection are mixed with 0.5 g of sodium disulphite, 3 g of n-butanol, 2.6 g of sodium chloride and 0.1 g of poloxamer. 2 pradofloxacin (trihydrate, calculated as pure pradofloxacin) are dissolved in this mixture. If required, a pH of 7.4 is set with approximately 2.4 g of sodium hydroxide, and the mixture is brought to the final volume of 100 ml with the remaining water for injection.

### In-Vivo Tolerance

In clinical trials, the formulations described herein have demonstrated an improved local tolerance in comparison with other formulations. The extent of tissue irritation and swelling at the injection site as the result of active substance depends on the formulation employed. Selected examples are listed in Table 1 which follows.

TABLE 1

| | Local reactions, dog | | | | |
|---|---|---|---|---|---|
| | 1-36 days post-administration | | | | |
| Formulation | n | spontaneous | mild | moderate | severe |
| Example 2 | 6 | 0 | 0 | 0 | 0 |
| 3% pradofloxacin with | | | | | |
| sodium disulphite (SC, | | | | | |
| 3 ml) | | | | | |
| Example 6 | 8 | 0 | 2 | 0 | 0 |
| 2% pradofloxacin with | | | | | |
| sodium disulphite (SC, | | | | | |
| 9 mg/kg) | | | | | |
| Example 2 | 6 | 0 | 2 | 0 | 0 |
| 3% pradofloxacin with | | | | | |
| sodium disulphite (SC, | | | | | |
| 9 mg/kg) | | | | | |
| Example 2 | 6 | 0 | 0 | 0 | 0 |
| 3% pradofloxacin with | | | | | |
| sodium disulphite (IM, | | | | | |
| 9 mg/kg) | | | | | |

SC = subcutaneous,
IM = intramuscular

### Serum-Pharmacokinetic Profile

The formulation affects the serum-pharmacokinetic (PK) profile. Different formulations differ markedly with regard to their serum concentration time-curve. Curves with rapid absorption, high peak concentrations and long elimination phases are preferred for quinolones. Table 2 hereinbelow lists various formulations and shows their influence on the PK profile.

TABLE 2

| | | PK parameters (arithmetic mean) | | | | |
|---|---|---|---|---|---|---|
| | | | | | AUC_{inf} | |
| | | Cₘₐₓ | Tₘₐₓ | t_{1/2} | (h * | MRTₗₐₛₜ |
| Formulation | n | (μg/ml) | (hr) | (hr) | μg/ml) | (hr) |
| Example 6 | 8 | 2.7 | 3.3 | 7.9 | 36.0 | 13.3 |
| 2% pradofloxacin with | | | | | | |
| sodium disulphite | | | | | | |
| (SC, 9 mg/kg) | | | | | | |
| Example 2 | 6 | 2.7 | 2.3 | 5.1 | 28.0 | 8.4 |
| 3% pradofloxacin with | | | | | | |
| sodium disulphite | | | | | | |
| (IM, 9 mg/kg) | | | | | | |

SC = subcutaneous,
IM = intramuscular

1. Pharmaceutical formulation in liquid form containing:
(a) a fluoroquinolone
(b) an antioxidative sulphur compound
(c) if appropriate, further pharmaceutical auxiliaries and/or additives.

2. Pharmaceutical formulation according to claim 1, containing, as antioxidative sulphur compound, a sulphite, in particular sodium sulphite, potassium sulphite; a bisulphite, in particular sodium metabisulphite, potassium metabisulphite, potassium pyrosulphite, sodium pyrosulphite, acetosodium metabisulphite, acetosodium bisulphite; a thiosulphate, in particular potassium thiosulphate, sodium thiosulphate; or an organic sulphur compound, in particular sodium formaldehyde sulphoxylate, thiourea, thiosorbitol, cysteine hydrochloride, cystine, cysteine, acetylcysteine, glutathione, cysteamine, methionine, thioglycerol, thioglycolic acid, thiolactic acid.

3. Pharmaceutical formulation according to one of the preceding claims, containing sodium disulphite.

4. Pharmaceutical formulation according to one of the preceding claims, containing, as fluoroquinolone, ciprofloxacin, enrofloxacin, pradofloxacin or marbofloxacin.

5. Pharmaceutical formulation according to claim 4, containing pradofloxacin.

6. Pharmaceutical formulation according to claim 4, containing enrofloxacin.

7. Pharmaceutical formulation according to claim 4, containing marbofloxacin.

8. Use of pharmaceutical formulations according to one of the preceding claims for the preparation of pharmaceuticals for controlling bacterial diseases.

9. Use according to claim 8 for the preparation of pharmaceuticals which are suitable for injection, for controlling bacterial diseases.

10. Use according to claim 8 or 9 for the preparation of pharmaceuticals which are better tolerated.
